(19) United States
(12) Patent Application Publication
(10) Pub. No.: US 2010/0331677 A1
(43) Pub. Date: Dec. 30, 2010

(54) MARKER DELIVERY SYSTEM

(75) Inventors: Kelvin K. Hong, Baltimore, MD (US); Ian Lee, Baltimore, MD (US); Pao-Lin Che, Baltimore, MD (US); Brian Hsi, Houston, TX (US); Linmiao Xu, Baltimore, MD (US); Deepika Sagaram, Baltimore, MD (US)

Correspondence Address: VENABLE LLP, P.O. BOX 34385, WASHINGTON, DC, 20043-9998, US

(73) Assignee: The Johns Hopkins University, Baltimore, MD (US)

(21) Appl. No.: 12/918,576

(22) PCT Filed: Apr. 27, 2009
(86) PCT No.: PCT/US09/41822

§ 371 Date: Aug. 20, 2010

Related U.S. Application Data
(60) Provisional application No. 61/125,527, filed on Apr. 25, 2008.

(51) Int. Cl. A61B 6/00

(52) U.S. Cl. 600/432

(57) ABSTRACT

A marker delivery system including a surgical needle defining a lumen. The surgical needle is adapted to receive a marker. The surgical needle includes a side opening substantially adjacent to a first end of the surgical needle. The marker delivery system also includes a plunger insertable into the surgical needle at a second end of the surgical needle. Depression of the plunger inside the lumen of the surgical needle pushes the marker through the side opening of the surgical needle.

### CROSS-REFERENCE TO RELATED APPLICATION

This application claims priority to U.S. Application No. 61/125,527 entitled “Marker Delivery System For External Beam Radiation Therapy,” filed Apr. 25, 2008 which is herein incorporated by reference in its entirety.

### BACKGROUND

The clinical success of External Beam Radiation Therapy (EBRT) for cancer is determined by accuracy of tumor identification, often achieved through use of implanted markers. Clinical success of EBRT is largely dependent on maximizing radiation towards the tumor and minimizing radiation towards the surrounding healthy tissue. This is crucial for successful treatment, as radiation commonly affects regions where it is not desired. The only possible way to minimize the harmful effects on healthy regions is to focus radiation towards the tumor site as precisely as possible. Current methods of focusing radiation can be classified into two categories: three-dimensional radiation therapy and four-dimensional radiation therapy, or Image-Guided Radiation Therapy (IGRT), which uses three-dimensional radiation therapy techniques as well as tracking of tumor location. Both therapy techniques introduce lower side effects when compared to the conventional broad-area radiation therapy. Four-dimensional radiation therapy techniques improve upon three-dimensional techniques by taking into account the possible changes in tumor location. This allows for adjustments to be made before or during actual treatment, and, as a result, radiation therapy can conform more closely to the tumor shape.

Physicians can currently focus the radiation field toward the tumor site with some precision, but can greatly increase accuracy if tumor movement is taken into account. Tumor movement occurs if the patient moves during treatment (such as breathing or shivering), or if the tumor changes shape over the course of treatment. Confining the radiation dose closer to the tumor shape significantly reduces the margin of error when compared to the conventional broad area radiation therapy, because any movement of the tumor can result in the radiation missing the target. Thus, in order to deliver the most effective form of EBRT, there must be a way to track the motion of the tumor during treatment. In current methods, tumor tracking is done by implanting a gold marker into or around the tumor site. The gold marker serves as a visible landmark by which machines can pinpoint where the tumor is at all times. Currently, marker implantation uses 17-gauge (1.47 mm outer diameter) needles.

The current 17-gauge of the needle poses two problems. First, the invasiveness of a large needle can lead to significant trauma. In low-risk areas, such as the prostate, this trauma can lead to delayed treatment. In high-risk areas, such as the lung or abdominal region, this trauma can lead to potentially life-threatening complications, ranging from collapsed lungs to organ failure. Second, the poor general health of many cancer patients prevents them from receiving many medical procedures, including marker implantation by current marker delivery needles. Studies show that the use of smaller needles can significantly reduce the incidence of complications during marker implantation, thus making effective EBRT more readily available to all cancer patients.

Conventional markers used for external beam radiation therapy must be large enough to be visible under CT imaging. Smaller needles, while having fewer harmful effects, implant smaller markers, which may not be visible under imaging. As a result, marker implantation is only used for a handful of patients today, with the vast majority being prostate cancer patients. It is nearly impossible with current technology to implant markers in high risk regions such as the lung or the gastrointestinal areas. More than 61% of all cancer patients cannot receive marker implantation because of resulting complications. This presents a great obstacle, as the more advanced and effective forms of EBRT, especially IGRT is dependent on the placement of CT visible markers as a form of reference in order to easily determine the location of the target tumor initially, as well as tracking in real time during therapeutic radiation. If markers cannot be placed, the patient either undergoes less effective EBRT or does not undergo EBRT at all. Therefore, there is a need for improved methods that allow more effective forms of EBRT to patients. The invasive nature of current methods renders marker implantation inaccessible for many patients. The present invention discloses a novel marker delivery system that uses a minimally invasive needle to safely implant markers into most areas of the body.

### SUMMARY

According to one embodiment of the invention, there is provided a marker delivery system, comprising: a surgical needle defining a lumen, the surgical needle adapted to receive a marker, wherein the surgical needle includes a side opening substantially adjacent to a first end of the surgical needle; and a plunger insertable into the surgical needle at a second end of the surgical needle. The surgical needle at least one of defines or comprises a ramp formed in the lumen proximate the side opening, and the depression of the plunger inside the lumen of the surgical needle pushes the marker to deflect from the ramp and to pass through the side opening of the surgical needle.

According to another embodiment of the invention, there is provided a marker delivery system, comprising: a surgical needle defining a lumen, the surgical needle adapted to receive a plurality of markers, wherein the surgical needle includes a side opening; and a plunger insertable into the surgical needle at a second end of the surgical needle, wherein depression of the plunger inside the lumen of the surgical needle pushes each marker sequentially through the side opening of the surgical needle.

### BRIEF DESCRIPTION OF THE DRAWINGS

The present invention will be more readily understood from the following detailed description when read in conjunction with the accompanying drawings, in which:

FIG. 1 is a detailed view of the surgical needle and needle handle;

FIG. 2 is a detailed view of the plunger with the plunger handle;

FIG. 3 is a detailed view of a cross-section of the needle tip;

FIG. 4 is a perspective view of the inside of the needle tip;

FIG. 5 is an overview of the plunger and needle according to an embodiment of the invention;

FIG. 6 is a view of the plunger with the plunger handle according to an embodiment of the invention;

FIG. 7 is a magnified view of the needle tip according to an embodiment of the invention;

FIG. 8 is a magnified view of the needle handle and staircase track according to an embodiment of the invention;

FIG. 9 is a view of the knob at the top of the second vertical section according to an embodiment of the invention;

FIG. 10 is a view of the knob at the bottom of the second vertical section according to an embodiment of the invention;

FIG. 11 is a view of the knob at the end of the second horizontal section according to an embodiment of the invention;

FIG. 12 is a view of the knob at the bottom of the third vertical section according to an embodiment of the invention;

FIG. 13 is a view of the knob at the end of the third horizontal section according to an embodiment of the invention;

FIG. 14 is a view of the knob at the bottom of the fourth vertical section according to an embodiment of the invention;

FIG. 15 is a view of the knob at the end of the fourth horizontal section according to an embodiment of the invention;

FIG. 16 is a view of the knob at the bottom of the fifth vertical section according to an embodiment of the invention;

FIG. 17 is a view of the knob at the end of the fifth horizontal section according to an embodiment of the invention;

FIG. 18 is a view of the knob at the bottom of the sixth vertical section according to an embodiment of the invention;

FIGS. 19A and 19B are images from an experiment with a single marker; and

FIGS. 20A and 20B are images from an experiment with a cluster of five markers using the delivery system according to an embodiment of the invention.

### DETAILED DESCRIPTION

An embodiment of the invention involves the delivery of a cluster of visible fiducial markers through a side opening at the tip of a needle, for example, using a 21-gauge surgical needle. A series of markers is pre-loaded sequentially inside the needle. Deployment of each marker may be controlled by a track mechanism at the needle base. The invention utilizes a side-deployment method of marker insertion. A ramp angled from the needle's side opening allows the markers to slide out of the needle lumen, and the plunger controls individual marker deployment. The needle is rotated after deploying each marker.

The visibility of a cluster composed of small markers is comparable to that of a single large marker, effectively delivering a large, visible marker through a minimally-invasive procedure. Experiments in an animal model confirm the efficacy of marker deployment as well as comparable marker visibility to current state of the art. The device according to embodiments of the invention differs from current marker delivery systems in that the delivery needle is much smaller, thus reducing invasiveness. The side delivery of multiple markers is also a novel method of effectively inserting a CT visible marker while using smaller markers and needle.

The device may implant a cluster of smaller markers. The markers may be equal to or larger than 0.5 mm in diameter and 5 mm in length. The cluster of markers visually simulates the effect of a single larger marker, for example a marker 1.2 mm×5 mm. Using a smaller marker allows a reduction in needle size, which in turn translates into a less invasive procedure. The delivery system according to some embodiments of the invention provides for a minimally-invasive delivery system with the needle approximately 0.8 mm in diameter to insert markers. This makes marker implantation a possibility for many more patients, and therefore more treatment through EBRT.

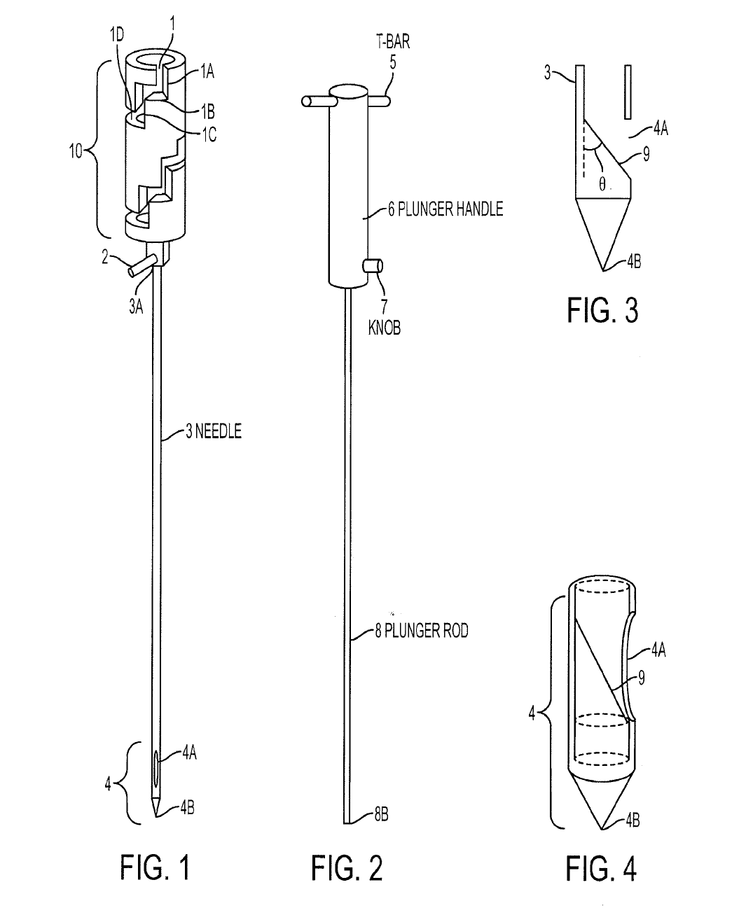

FIGS. 1 and 2 illustrate an embodiment of a marker delivery system comprising a surgical needle 3 with a needle handle 10 and a plunger 8 with a plunger handle 6. The surgical needle can be stainless steel or it can be a Magnetic Resonance Imaging (MRI) compatible material. The needle handle 10 can be plastic or any other comparable material. According to an embodiment of the invention, the surgical needle 3 minimizes procedure invasiveness owing to the reduction in needle size in comparison to needles currently used in fiducial marker implantation procedures. An example of such surgical needle 3 is a 21-gauge stainless steel needle with an inner diameter of 0.5 mm and an outer diameter of 0.8 mm. The reduction in needle size is made possible by the use of markers of smaller size. However, the invention is not limited to only 21-gauge surgical needles.

The surgical needle 3 houses markers in the lumen of the surgical needle 3. The diameter of the markers can be relatively small, for example, approximately 0.5 mm in some embodiments. The markers can be composed of a biologically inert substance. An example of a substance that is suitable for some embodiments is 14K gold, which is visible under imaging. The markers can be preloaded in the surgical needle 3 to reduce the procedure time. Examples of marker companies that produce markers similar to the markers used according to the invention, include, but are not limited to IZI Medical Product, ONC Solutions, CIVCO Medical Solutions, and Core Oncology.

As shown in FIG. 1, the surgical needle 3 includes a needle tip 4. The needle tip 4 includes a side opening 4A and a beveled end point 4B located beneath the side opening 4A. The needle tip 4 may be visible in tissue by utilizing the marker delivery system with an imaging apparatus in order to facilitate guidance of the surgical needle 3 to the target. Examples of such imaging apparatuses include, but are not limited to ultrasonography, magnetic resonance imaging (MRI), and computed tomography.

The length of the surgical needle 3 defines a longitudinal direction. The width of the surgical needle is transverse to the longitudinal direction. The width center of the surgical needle defines an axis of rotation for the surgical needle that is parallel to the surgical needle length. The surgical needle 3 may rotate about this axis. The height of the side opening extends along the longitudinal direction of the surgical needle.

In an embodiment, the side opening 4A may have a height less than the height of a single marker, for example, 3 mm. The side opening 4A reduces the possibility of the marker falling out when the needle is withdrawn during the middle of a procedure. As shown in FIGS. 3 and 4, a cross section of the needle tip 4 shows a ramp 9 extending from the base of the side opening 4a and reaching up to the opposing interior wall of the surgical needle 3. The angle θ may define the angle of depression of the ramp 9. As shown in FIG. 3, angle θ may be an acute angle, for example, 30 degrees or less.

The surface of the ramp 9 may be embodied as substantially linear from top to bottom, may be slightly curved, or may be stepwise linear, but not limited to those embodiments. The base of the ramp may be flush with the bottom of the side opening 4A. The area beneath the ramp 9 to the end point 4B may be a solid metal body. The end point 4B may be pyramidal in shape and the surface of the needle tip 4B may be ridged for improved ultrasonography visibility.

As shown in FIG. 2, the plunger 8 is coupled to the plunger handle 6. The plunger 8 can be stainless steel or it can be an MR compatible material. The plunger handle 6 can be plastic or any other comparable material. The diameter of the plunger handle 6 is less than the diameter of the needle handle 10 and the plunger handle 6 fits within the needle handle so that it may move smoothly along the track when force is applied and still maintain its current position when force is not applied 10. The width of the plunger 8 is accordingly less than the width of the surgical needle 3.

The plunger handle 6 includes a knob 7 located at the base end of the plunger handle 6 which is closest to the plunger 8 connection. The knob 7 is substantially perpendicular to the plunger 8. The shape of the knob 7 may be a variety of shapes including a cylindrical or a rectangular prism shape. The knob 7 locks the plunger handle 6 with the needle handle 10. A T-bar 5 is located at the opposed end of the handle away from the knob. The T-bar 5 intersects the plunger handle 6 and is substantially perpendicular to the plunger 8. The T-bar assists in pushing the plunger in a descending direction starting from the T-bar 5 towards the knob 7. The T-bar also may assist in keeping the plunger handle 6 steady as the surgical needle 3 via the needle handle 10 rotates.

The needle handle 10 may include a staircase track 1. The staircase track 1 has a width greater than the width of the knob so that the knob 7 may be insertable in the track 1. In another embodiment of the invention, the track is embodied as a threaded screw system. In order to facilitate expedient implantation of the marker, the knob 7 and the staircase track 1 are designed to allow for discrete movement of the plunger 8 down the inside of the surgical needle 3, as well as discrete rotation of the surgical needle 3. The plunger 8 may be insertable into the surgical needle 3 via the needle handle 10. Likewise, the plunger handle 6 is insertable in the needle handle 10.

The knob 7 follows the track 1 so that the plunger 8 advances toward the tip 4 of the needle. Once the plunger 8 approaches the last marker of the series of markers, the plunger 8 may bend at the interface of the ramp 9 in a flexion region to deliver the marker through the side opening 4A into tissue. The plunger 8 functions to push the marker, preloaded inside the needle, out the side opening 4a of the needle.

The track 1 includes a series of vertical sections and horizontal sections. When the knob enters the first vertical section 1A and the plunger is depressed so that the knob reaches the base of the first vertical section, the marker that is located at the first end of the surgical needle 3, is placed in position to be deposited through the side opening 4A by the plunger base 8B.

The plunger handle 6 and correspondingly the plunger 8, do not rotate in a rotational manner. The plunger handle 6 and the plunger 8 move in a direction toward the tip 4 of the surgical needle 3. In contrast, the needle handle 10 and the surgical needle 3 rotate rotationally in order to rotate the location of the side opening 4A. The vertical sections of the staircase track limit plunger movement only to increments which correspond to the length of the markers. At the same time, the horizontal sections of the staircase track limit rotation of the needle handle 10, if the plunger handle 6 is held steady, to discrete angular rotations, such that a plurality of markers will be deployed in a substantially symmetrical circular fashion.

In positioning the first marker for deposition into tissue, the needle handle 10 is rotated so that the knob 7 moves along the first horizontal path 1B. The first horizontal section 1B causes the rotation of the needle handle 10 and surgical needle 3 correspondingly. The second vertical section 1C first deploys the marker out of the side opening 4A and into tissue. The second horizontal section 1D corresponds to the rotation of the needle 3 by a specified angle, for example 72 degrees to deploy 5 markers. After the second vertical track 1C, each successive vertical section corresponds to deployment of one marker, whereas each successive horizontal section corresponds to the rotation of the needle by the specified angle.

The height of each vertical track corresponds to at least the height of the marker, for example 7 mm. The height ensures that only one marker will be ejected at a time. The base of each vertical section, or beginning on each horizontal section, on the staircase track 1 has a small depression in height of approximately less than 1 mm. The depression prevents horizontal sliding of the knob after each marker in the needle is deposited out of the side opening 4A.

In an embodiment, the staircase track includes at least three sets of stairs, with the length of each horizontal section directly related to the preferred angle of needle rotation, and height of each vertical section greater than or equal to the length of each individual marker. The staircase track 1 interfaces with the knob 7 on the plunger handle 6 to control ejection of individual markers via the side opening 4A.

A peg 2 is located at the connection of the needle handle with the top 3A of the surgical needle 3. The peg 2 is substantially perpendicular to the surgical needle 3 and matches the direction of the side opening 4A. The peg may be used to assist in rotation of the needle handle 10 to move the knob 7 along a horizontal section of the staircase track 1. The peg 2 may also be used to judge the location of the side opening 4A in order to determine the angle of marker placement prior to the marker deposition.

The marker delivery system may be used to insert the markers via the surgical needle 3 into a patient. During needle insertion, the end point 4B of the surgical needle 3 may be tracked using an imaging apparatus until the needle is within the target tumor. Once the needle is in the proper place for marker depositing, an operator of the marker delivery device may depress the plunger handle to move the knob 7 one vertical step of the staircase track 1, as shown in FIG. 10. After one marker is inserted into the tumor tissue, the needle handle 10 and surgical needle 3 may be rotated to move the knob 7 along the horizontal section of the track 1, as shown in FIG. 11. The rotation of the surgical needle 3 causes the side opening 4A to face a new direction. Once rotated, another marker may be inserted into the tumor tissue by depressing the plunger handle 6 which moves the knob 7 along the next vertical section of the track 1, as shown in FIG. 12. This process may be repeated until a cluster of markers is formed. When all markers are inserted, they form a cone structure, which is designed to simulate a single larger marker under CT scan.

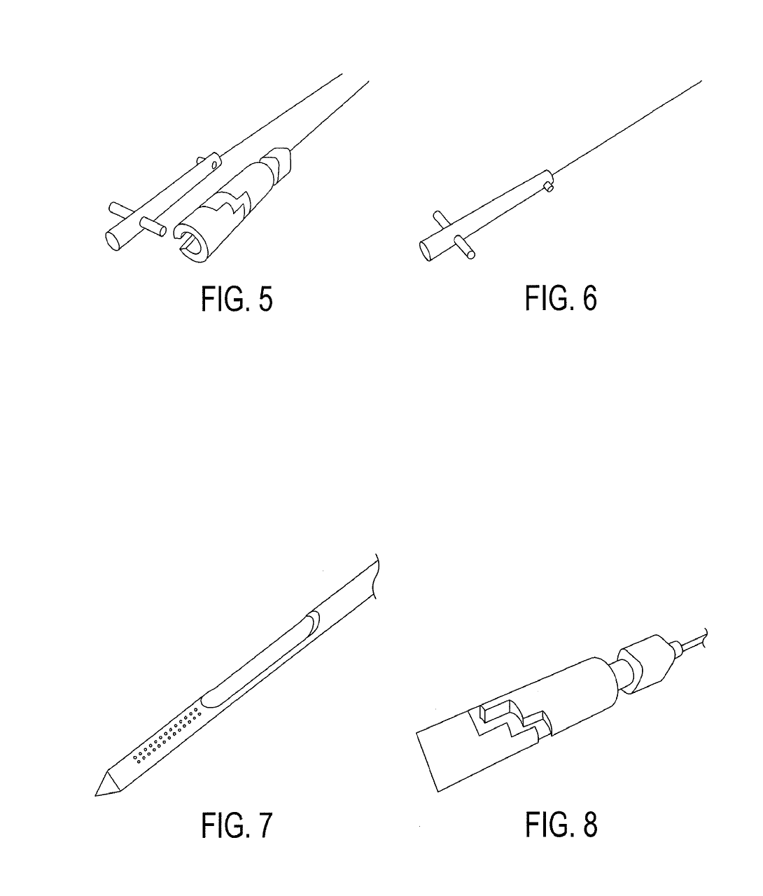

FIG. 5 shows the plunger 8 with plunger handle 6 separated from the surgical needle 3 with needle handle 10.

FIG. 6 shows a magnified view of the plunger 8 with plunger handle 6.

FIG. 7 shows a magnified view of the needle tip 4. The side opening 4A is shown so that the ramp 9 is visible. FIG. 7 shows that the area from the base of the side opening 4A to the end point 4B is beveled.

FIG. 8 shows a magnified view of the needle handle 10 with the staircase track 1.

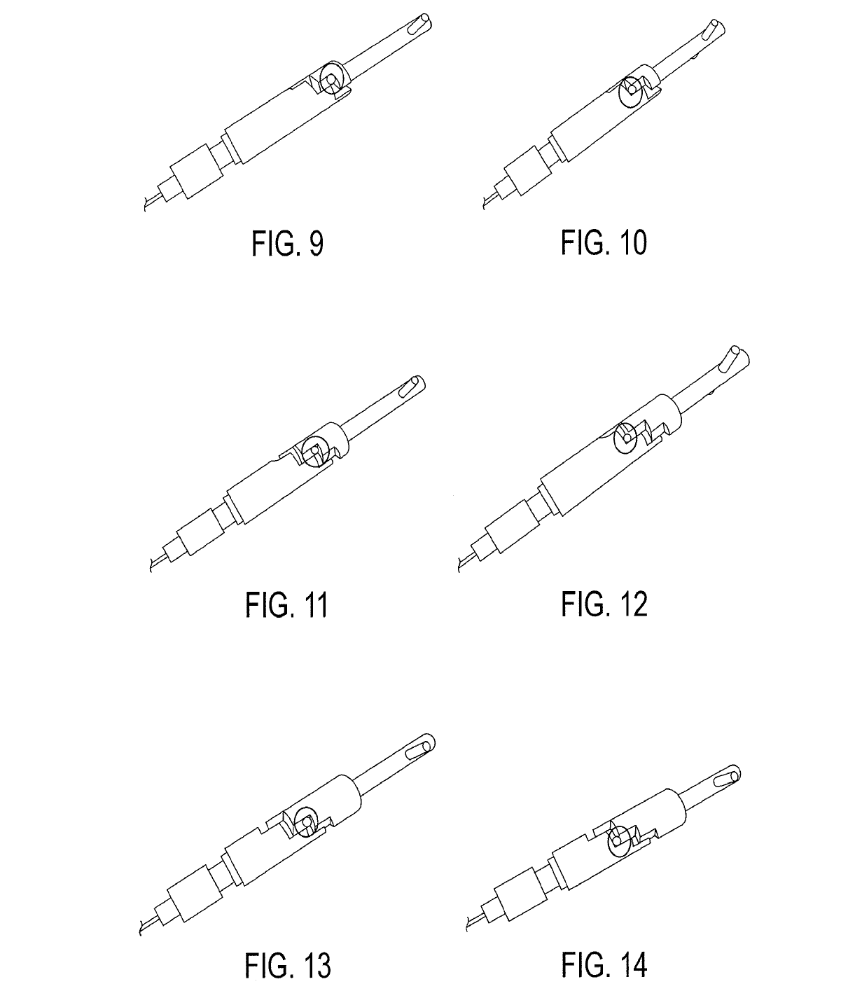

FIG. 9 shows the plunger 8 inserted into the surgical needle 3. Likewise, the plunger handle 6 is inserted into the needle handle 10. Accordingly, the knob 7 is within the staircase track 1 and locks the plunger handle 6 to the needle handle 10. FIG. 9 displays the knob 7 at the top of the second vertical step which is prior to the implantation of a first marker.

FIG. 10 shows the next phase in the operation of the marker delivery system. The plunger handle 6 has been depressed so that the knob 7 is currently at the bottom of the second vertical section 1C.

FIG. 11 shows a subsequent phase from the phase shown in FIG. 10. In FIG. 11, the needle handle 10 has been rotated so that the knob 7 is located at the end of the second horizontal section 1d. At this phase, a second marker is in position for depositing into tissue.

FIG. 12 shows a subsequent phase from FIG. 11. FIG. 12 shows that the plunger handle 6 has been depressed so that the knob 7 is at the base of the third vertical section. Accordingly, a second marker was deposited with the depression of the plunger handle 6.

FIG. 13 shows a next phase from FIG. 12. In FIG. 13, the needle handle 10 has been rotated so that the knob 7 is located at the end of the third horizontal section. At this phase, a third marker is in position for depositing into tissue.

FIG. 14 shows a subsequent phase from FIG. 13. FIG. 14 shows that the plunger handle 6 has been depressed so that the knob 7 is at the base of the fourth vertical section. Accordingly, a third marker was deposited with the depression of the plunger handle 6.

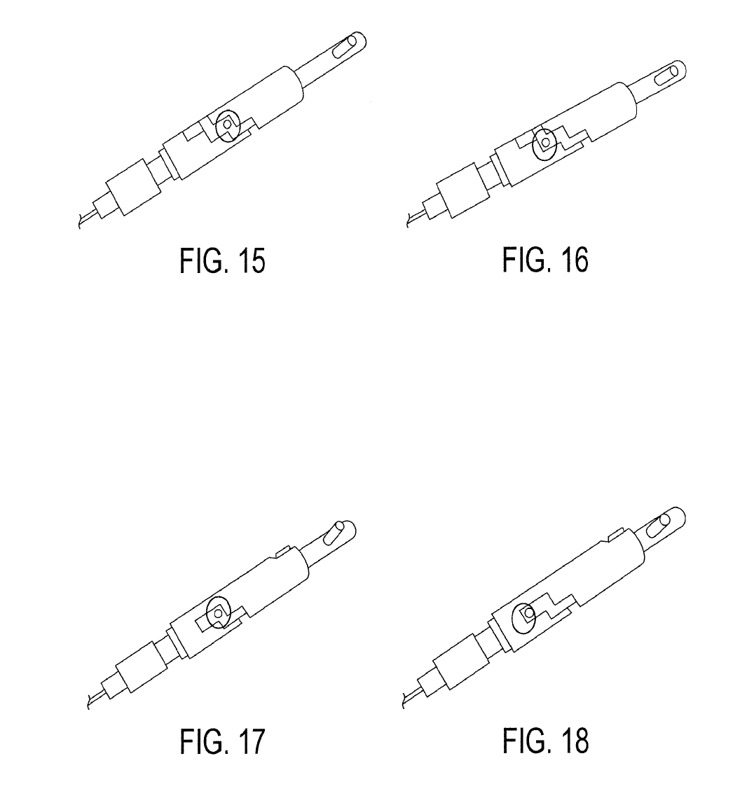

FIG. 15 shows a subsequent phase from the phase shown in FIG. 14. In FIG. 15, the needle handle 10 has been rotated so that the knob 7 is located at the end of the fourth horizontal section. At this phase, a fourth marker is in position for depositing into tissue.

FIG. 16 shows a subsequent phase from FIG. 15. FIG. 16 shows that the plunger handle 6 has been depressed so that the knob 7 is at the base of the fifth vertical section. Accordingly, a fourth marker was deposited with the depression of the plunger handle 6.

FIG. 17 shows a subsequent phase from the phase shown in FIG. 16. In FIG. 17, the needle handle 10 has been rotated so that the knob 7 is located at the end of the fifth horizontal section. At this phase, a fifth marker is in position for depositing into tissue.

FIG. 18 shows a subsequent phase from FIG. 17. FIG. 18 shows that the plunger handle 6 has been depressed so that the knob 7 is at the base of the sixth vertical section. Accordingly, a fifth marker was deposited with the depression of the plunger handle 6. The track may have as many steps in the track as necessary and the track is not limited to the number of steps described in any particular embodiment of the invention.

In experimenting with the marker delivery system, two sets of markers were implanted into the liver of a recently deceased pig. One set comprised five (0.5 mm×5 mm) 14 K gold markers. The five markers were delivered using the delivery system under ultrasonography guidance. The second marker set was one (0.5 mm×5 mm) 14K gold marker. The one marker was delivered using a standard 21 gauge needle under ultrasonography guidance. The subsequent CT scan showed that the five marker cluster actually showed greater visibility than both the image background and the one individual marker.

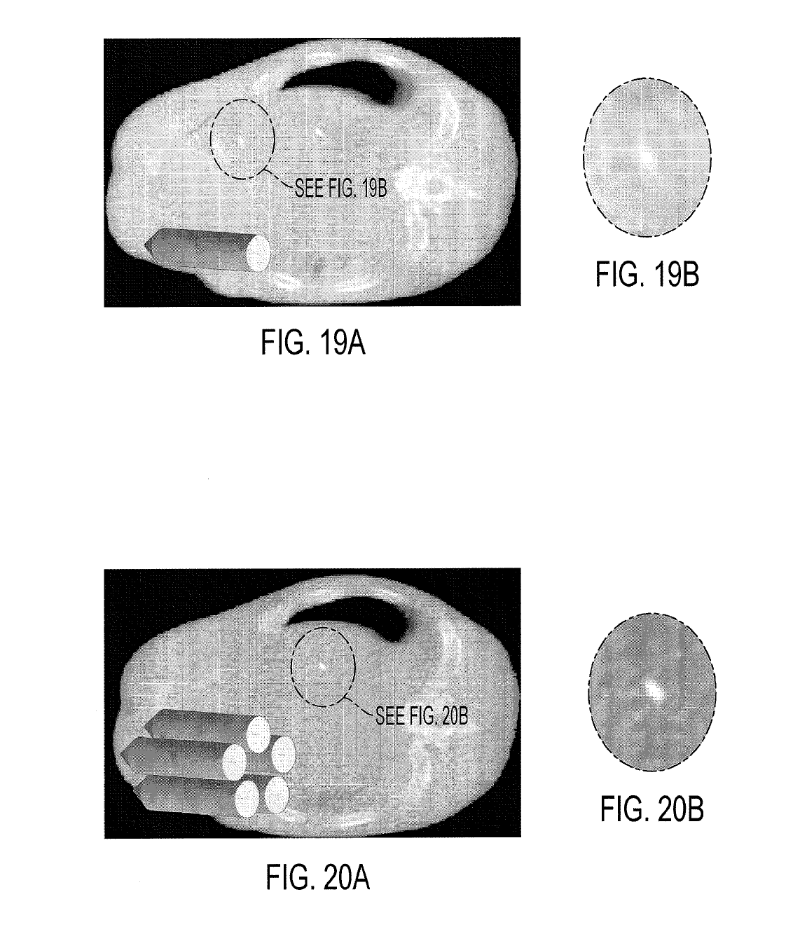

Using the CT scan grayscale as a means of quantitative comparison, the image of the one marker was estimated to be approximately 17% brighter than the average image background. The percentage was determined by comparing the peak grayscale value in the marker versus the average image background. The results of the imaging are shown in FIGS. 19A and 19B. FIG. 19B is a magnified representation of the marker image in the circle of FIG. 19A.

In contrast, the cluster of the five smaller markers had approximately 38% more attenuation than the average image background. The results of the imaging are shown in FIGS. 20A and 20B. FIG. 20B is a magnified representation of the marker cluster in the circle of FIG. 20A. Thus, the cluster of five markers was able to approximately double the visibility of the existing marker design while reducing procedure invasiveness.

In other embodiments of the invention, a variety of kinds of markers may be used such as, but not limited to, biocompatible material, composite elements, or elements with high atomic numbers. In addition, the marker delivery system may be embodied so that varying numbers of markers may be used for a cluster. The marker delivery system may also be automated.

It will be understood that the above description of the present invention is susceptible to various modifications, changes and adaptations, and the same are intended to be comprehended within the meaning and range of equivalents of the appended claims.

We claim:

1. A marker delivery system, comprising:
a surgical needle defining a lumen, the surgical needle adapted to receive a marker, wherein the surgical needle includes a side opening substantially adjacent to a first end of the surgical needle; and
a plunger insertable into the surgical needle at a second end of the surgical needle,
wherein the surgical needle at least one of defines or comprises a ramp formed in said lumen proximate said side opening, and
wherein depression of the plunger inside the lumen of the surgical needle pushes the marker to deflect from the ramp and to pass through the side opening of the surgical needle.

2. A marker delivery system according to claim 1, further comprising:
a needle handle coupled to a second end of the surgical needle, the needle handle including a track; and
a plunger handle coupled to the plunger, the plunger handle including a knob, wherein upon insertion of the plunger into the surgical needle, the knob follows the track of the needle handle to deposit the marker through the side opening of the surgical needle.

3. The marker delivery system according to claim 2, wherein the track has a staircase configuration, the staircase configuration comprising alternating vertical and horizontal sections substantially perpendicular to each other.

4. The marker delivery system according to claim 3, wherein the staircase configuration includes a first vertical section that corresponds to a ready position of the marker and a second vertical section that corresponds to a deposition of a marker through the side opening of the surgical needle.

5. The marker delivery system according to claim 3, wherein the needle handle rotates about an axis of rotation for the surgical needle to shift the knob along each horizontal section so that the side opening of the surgical needle rotates correspondingly.

6. The marker delivery system according to claim 3, wherein each vertical section after the first vertical section corresponds to a height of the marker.

7. The marker delivery system according to claim 3, wherein each horizontal section corresponds to a predetermined degree of rotation.

8. The marker delivery system according to claim 3, wherein the plunger handle moves vertically along the track toward the first end of the surgical needle to deposit the marker as the needle handle remains substantially stationary.

9. The marker delivery system according to claim 1, wherein the first end of the surgical needle further comprises an end point, wherein the side opening is located above the end point, and said ramp located inside the needle lumen is flush with a base of the side opening and extends at an angle inside the lumen to an inside opposing wall of the surgical needle with respect to the side opening, and the ramp guides the marker through the side opening upon application of a force by the plunger.

10. The marker delivery system according to claim 1, wherein the surgical needle is smaller than a 17-gauge surgical needle.

11. The marker delivery system according to claim 1, further comprising:
a marker, wherein a width of the marker is less than a width of the surgical needle and the marker is depositable into the lumen.

12. The marker delivery system according to claim 11, wherein the marker comprises gold.

13. A marker delivery system, comprising:
a surgical needle defining a lumen, the surgical needle adapted to receive a plurality of markers, wherein the surgical needle includes a side opening; and
a plunger insertable into the surgical needle at a second end of the surgical needle, wherein depression of the plunger inside the lumen of the surgical needle pushes each of the plurality of markers sequentially through the side opening of the surgical needle.

14. The marker delivery system according to claim 13, wherein the surgical needle comprises a first end, the first end of the surgical needle comprising:
an end point, wherein the side opening is located above the end point, and
a ramp located inside the needle lumen, wherein the ramp is flush with a base of the side opening and extends at an angle inside the lumen to an inside opposing wall of the surgical needle with respect to the side opening, and the ramp guides the marker through the side opening upon application of a force by the plunger.

15. A marker delivery system according to 13, further comprising:
a needle handle coupled to the second end of needle, the needle handle including a track; and
a plunger handle coupled to the plunger, the plunger handle including a knob, wherein upon insertion of the plunger into the surgical needle, the knob follows the track of the needle handle to rotate the surgical needle and to deposit each marker sequentially through the side opening of the surgical needle in a substantially circular cluster of markers.

16. The marker delivery system according to claim 15, wherein the track has a staircase configuration, the staircase configuration comprising alternating vertical and horizontal sections substantially perpendicular to each other.

17. The marker delivery system according to claim 16, wherein a first vertical section corresponds to a ready position of the marker and a second vertical section corresponds to a deposition of a marker through the side opening of the surgical needle.

18. The marker delivery system according to claim 16, wherein the plunger handle moves vertically along the track toward a first end of the surgical needle to deposit the marker as the needle handle remains substantially stationary.

19. The marker delivery system according to claim 16, wherein the needle handle rotates about an axis of the surgical needle to shift the knob along each horizontal section so that the side opening of the surgical needle rotates correspondingly.

20. The marker delivery system according to claim 16, wherein each vertical section after the first vertical section corresponds to a height of the marker.

21. The marker delivery system according to claim 16, wherein each horizontal section corresponds to a predetermined degree of rotation.

22. The marker delivery system according to claim 13, wherein the surgical needle is smaller than a 17-gauge surgical needle.

23. The marker delivery system according to claim 13, further comprising:
a marker, wherein the marker is less than a width of the surgical needle and the marker depositable into the lumen.

24. The marker delivery system according to the claim 23, wherein the marker comprises gold.
